(19) United States
(12) Patent Application Publication
(10) Pub. No.: US 2008/0113042 A1
(43) Pub. Date: May 15, 2008

(54) PHARMACEUTICAL COMPOSITION AND METHOD FOR CANCER TREATMENT BASED ON COMBINATIONAL USE OF CONVENTIONAL ANTICANCER AGENTS AND GERANIUM OIL OR COMPOUNDS THEREOF

(76) Inventors: Kee Hung CHU, Hong Kong (CN); Wei Dong Xie, Hong Kong (CN); Cheong Yip Ho, Hong Kong (CN)

Correspondence Address: EVAN LAW GROUP LLC, 600 WEST JACKSON BLVD., SUITE 625, CHICAGO, IL, 60661, US

(21) Appl. No.: 11/850,155

(22) Filed: Sep. 5, 2007

Related U.S. Application Data
(60) Provisional application No. 60/824,464, filed on Sep. 5, 2006.

(51) Int. Cl. A61K 36/254; A61K 36/53; A61K 36/537; A61K 36/899; A61K 36/906; A61K 36/48; A61K 36/00; A61K 36/258; A61K 36/23; A61K 36/185

(52) U.S. Cl. 424/725; 424/728; 424/756; 424/757; 424/764; 424/745; 424/746; 424/750; 424/769

(57) ABSTRACT

A cancer treatment composition includes geranium oil or its chemical constituents and a chemotherapeutic agent or plant extract selected from the group consisting of plant-derived bioactive compounds. The cancer is selected from the group consisting of lung, breast, ovary, prostate, colon, liver, gastrointestinal, head-and-neck, cervix, kidney, neuroblastoma, leukemia, lymphoma, and melanoma.

### CROSS REFERENCE TO RELATED APPLICATION

Pursuant to 35 U.S.C. 119(e), this application claims priority to U.S. Provisional Application No. 60/824,464, filed Sep. 5, 2006, the contents of which are hereby incorporated herein by reference.

### BACKGROUND

Cancer or neoplasm is a malignant growth, which is characterized by unregulated proliferation of cells in the body system. It can arise in any organ of the body such as lung, breast, ovary, intestine, leukocytes, etc. Cancerous cells propagate from a single cell and multiply without control to develop into tumor tissues. These cancerous cells can invade nearby tissues and can spread through the bloodstream and lymphatic system to other parts of the body (metastasis).

Most cancers can be treated, and some cured, depending on the specific type, location, and stage. Once diagnosed, cancer is usually treated with a combination of surgery, chemotherapy and radiotherapy. As research develops, treatments are becoming more specific for the type of cancer pathology. The majority of chemotherapeutic drugs can be divided into: alkylating agents (e.g. cyclophosphamide), antimetabolites (e.g. fluorouracil), plant alkaloids (e.g. paclitaxel), topoisomerase inhibitors (e.g. topotecan), and cytotoxic antibiotics (e.g. daunorubicin). Nygren, P (2001) Acta Oncol 40(2/3):166-174. All of these drugs impair cell division or DNA synthesis and functions. However, most of the chemotherapeutic drugs cause undesirable systemic effects such as cardiac or renal toxicity, marrow aplasia, alopecia, nausea and vomiting. Many researchers have tried to eliminate these side effects by increasing the availability of the drug to the tumor site. In recent years, considerable emphasis has been given to the development of new formulations of chemotherapeutic agents on the basis of drug delivery, in order to address issues such as poor drug solubility and high toxicity. Chemotherapeutic agents commonly used in cancer treatment, such as paclitaxel and fluorouracil, thus become the candidates for drug reformulation.

Paclitaxel is one of the promising chemotherapeutic agents, with a wide spectrum of activity against cancers of the breast, ovary, lung, esophagus and lymphomas. It exerts antitumor activity by binding to tubulin and stabilizing microtubules, thus blocking cell mitosis at the G2/M phase of cell cycle. The drug is a naturally occurring taxane-type diterpene isolated from the bark of the Pacific yew tree, Taxus brevifolia, which is a plant belonging to genus Taxus, family Taxaceae. It has been sold under the trade name Taxol® (Bristol-Myers Squibb) since 1993. The drug is only administered intravenously, since it is orally inactive due to membrane transport and liver metabolism limitations. Taxol is currently formulated in a vehicle containing approximately a 1:1 v/v mixture of polyoxyethylated castor oil (Cremophor EL) and ethanol. Cremophor EL, a commonly used surfactant for lipophilic compounds, has been associated with bronchospasms, hypotension, and other manifestations of hypersensitivity particularly following rapid administration. Long infusion times upon a 10-fold dilution of the ethanol:Cremophor EL solution and premedication are therefore required to reduce the adverse effects. It is thus apparent that there is a need for new formulations of paclitaxel that are more efficacious than the commercial product, and that can alleviate the issues of drug administration. Panayiotis, P et al. (2000) Pharm Res 17(2):175-182; Han, J et al. (2004) Pharm Res 21(9):1573-1580.

Fluorouracil (5-FU) is another commonly used chemotherapeutic drug for the treatment of cancer, particularly gastrointestinal, colorectal, head-and-neck, and breast cancers. It is a pyrimidine analogue which belongs to the antimetabolite family of drugs. The drug is transformed inside the cell into different cytotoxic metabolites, which are then incorporated into DNA and RNA, thus inducing cell cycle arrest and apoptosis. Efficacy of 5-FU is markedly limited due to its rapid degradation into 5,6-dihydro-5-fluorouracil via action of dihydropyrimidine dehydrogenase in the liver or in tumors. Therefore, continuous infusion of 5-FU over prolonged periods is required to enhance the drug bioavailability and antitumor activity. However, toxicities including myelosuppression, oral mucositis, and gastrointestinal toxicities such as diarrhea, stomatitis, nausea, and vomiting are still observed. There is a need to develop new preparations of 5-FU for reducing toxicity and/or increasing anticancer efficacy. Paek, S H et al. (2006) Biol Pharm Bull 29(5):1060-1063.

Due to the growing concern on the side effects caused by chemotherapeutic agents in cancer treatment, plant-derived extracts, such as essential oils, have become a focus as complementary or alternative medicine. Recent studies have suggested that plant essential oils can be used us a potential treatment for cancers. For example, essential oil extracted from the dried pericarp of Zanthoxylum schinifolium was found to induce apoptotic cell death of human hepatoma cells in vitro and in vivo. Paik, S Y et al. (2005) Biol Pharm Bull 28(5):802-807. Another example is Sophora flavonoids, including kurarinone, sophoraflavanone G, and kuraridin, isolated from Sophora flavescens. These flavonoids have shown potent glycosidase- and tyrosinase-inhibitory activities, and might in turn be closely related to cytotoxic effects towards cancer cells. Kim, J H et al. (2006) Biol Pharm Bull 29(2):302-305; Kim, S J et al. (2003) Biol Pharm Bull 26(9):1348-1350.

Geranium oil is a plant essential oil collected by steam distillation from the stem and leaves of the plant of division Magnoliophyta, class Magnoliopsida, order Geraniales, family Geraniaceae, and genus Pelargonium. There are about 700 varieties of the plant, but only 10 of them can supply the essential oil in viable quantities. Pelargoniums are now grown, and geranium oil is now produced, mainly in Algeria, Egypt, China, and Australia. Geranium oil includes various chemical constituents, including geraniol, geranyl formate, citronellol, citronellyl formate, linalool, eugenol, myrtenol, terpineol, citral, methone and sabinene. Geranium oil is widely used as a natural flavor additive for food, as a fragrance in perfumery, in aroma therapy, and in alternative medicines. Dietary monoterpenes are the major constituents responsible for the distinctive fragrance of geranium oil. Recent studies have demonstrated that monoterpenes in plant essential oil exert antitumor activities. Duncan, R E et al. (2004) Biochem Pharmacol 68:1739-1747. For example, geraniol, an acyclic monoterpene has in vitro and in vivo antitumor activity against murine leukemia, hepatoma, and melanoma cells. Crowell, P L (1999) J Nutr 129:775 S-778S; He, L et al. (1997) J Nutr 127:668-674. In addition, geraniol has been shown to sensitize human colon cancer cells to 5-FU treatment, possibly by perturbation of cell membrane permeability and signal transduction pathway. Carnesecchi, S et al. (2001) J Pharmacol Exp Ther 298(1): 197-200; Carnesecchi, S et al. (2002) J Pharmacol Exp Ther 301(2):625-630; Carnesecchi, S et al. (2002) J Pharmacol Exp Ther 303(2):711-715.

Accordingly, there is a vast need for novel pharmaceutical compositions containing bioactive substances isolated from natural resources, preferably plant extracts, which may be effectively used in the chemotherapeutic treatment of cancers.

### SUMMARY

According to one aspect, a novel pharmaceutical composition exhibiting anticancer activities for the treatment of cancer includes geranium oil or its chemical constituents obtained from plants (for example, from Pelargonium species), and chemotherapeutic agents. The composition may further include other plant extract or bioactive compounds extracted from plants. The composition may synergistically enhance the therapeutically effects of the chemotherapeutic agents.

According to another aspect, there are provided methods for using such novel pharmaceutical compositions for therapeutically treating human cancers. The composition may be used in combination with a pharmaceutically acceptable carrier.

Additional aspects and attendant advantages will be set forth, in part, in the description that follows, or would be readily apparent to one skilled in the art. The aspects and advantages may be realized and attained by means of instrumentalities and combinations particularly recited in the appended claims. It is to be understood that the foregoing general description and the following detailed description are exemplary and explanatory only and are not to be viewed as being restrictive, as claimed.

The various features of novelty which characterize the invention are pointed out with particularity in the claims annexed to and forming a part of this disclosure. For a better understanding of its operating advantages and specific objects attained by its use, reference should be made to the drawings and the following description in which there are illustrated and described preferred embodiments.

### BRIEF DESCRIPTION OF THE DRAWINGS

FIG. 1 shows the changes in tumor size of the breast tumor-bearing nude mice injected with a preferred composition comprising paclitaxel and geraniol.

### DETAILED DESCRIPTION

Reference will now be made in detail to a particular embodiment of the invention, examples of which are also provided in the following description.

Furthermore, it should be understood that the invention is not limited to the precise embodiments described below, and that various changes and modifications thereof may be effected by one skilled in the art without departing from the spirit or scope of the invention. For example, elements and/or features of different illustrative embodiments may be combined with each other and/or substituted for each other within the scope of this disclosure and appended claims. In addition, improvements and modifications which may become apparent to persons of ordinary skill in the art after reading this disclosure, the drawings, and the appended claims are deemed within the spirit and scope of the present invention.

All patents, patent applications and literatures cited in this description are incorporated herein by reference in their entirety. In the case of inconsistencies, the present disclosure, including definitions, will prevail.

The following definitions are applicable. The term chemotherapeutic agent herein refers to any substance capable of reducing or preventing the growth, proliferation, or spread of a cancer cell, a population of cancer cells, tumor, or other malignant tissue. The term is intended also to encompass any antitumor or anticancer agent.

When used herein, the term “cancer cell” is intended to encompass definitions as broadly understood in the art. In an embodiment, the term refers to an abnormally regulated cell that can contribute to a clinical condition of cancer in a human or animal. In an embodiment, the term can refer to a cultured cell line or a cell within or derived from a human or animal body. A cancer cell can be of a wide variety of differentiated cell, tissue, or organ types as is understood in the art.

The following abbreviations are applicable: GI50, 50% inhibition of cell growth (the concentration needed to reduce the growth of treated cells to half that of untreated [i.e., control] cells); TGI, 100% (total) growth inhibition (the concentration required to completely halt the growth of treated cells); LC50, 50% cell kill, or lethal concentration (the concentration that kills 50% of treated cells); IC50, the concentration of an inhibitor that is required for 50% inhibition of its target (it measures how much of a particular substance/molecule is needed to inhibit some biological process by 50%).

Novel pharmaceutical compositions may exhibit anticancer activities for the treatment of cancer, including lung, breast, ovary, prostate, colon, liver, gastrointestinal, head-and-neck, kidney, neuroblastoma, leukemia, lymphoma, and melanoma. The composition may include geranium oil or its chemical constituents obtained from plants including Pelargonium species; and chemotherapeutic agent, plant extract or bioactive compounds extracted from plants. For example, the composition may include paclitaxel, chemotherapeutic drugs (e.g. 5-FU, cisplatin), matrine, plant extract, or plant-derived bioactive compounds.

Geranium oil may be collected by steam distillation from the stem and leaves of the plant of division Magnoliophyta, class Magnoliopsida, order Geraniales, family Geraniaceae, and genus Pelargonium. Preferably, geranium oil is extracted from Pelargonium graveolens or Pelargonium capitatum grown in Kunming City of the Yunan Province in China. Examples of the major constituents of geranium oil may include citronellol, geraniol, geranyl formate, and citronellyl formate, linalool, eugenol, myrtenol, terpineol, citral, methone, and sabinene.

Geranium oil and Sophora extract may show synergistic inhibitory effect on gastric, lung, breast and prostate cancers. Geranium oil is claimed as safe food supplement in FDA list for human consumption, and may act as a carrier or enhancer when combined with an anticancer compound. Geraniol and citronellol are the major active constituents in geranium oil.

The chemotherapeutic agents or derivatives thereof may include cyclophosphamide, chlorambucil, melphalan, mechlorethamine, ifosfamide, busulfan, lomustine, streptozocin, temozolomide, dacarbazine, cisplatin, carboplatin, oxaliplatin, procarbazine, uramustine, methotrxate, pemetrexed, fludarabine, cytarabine, fluorouracil, floxuridine, gemcitabine, capecitabine, vinblastine, vincristine, vinorelbine, etoposide, paclitaxel, docetaxel, doxorubicin, daunorubicin, epirubicin, idarubicin, mitoxantrone, bleomycin, mitomycin, hydroxyurea, topotecan, irinotecan, amsacrine, teniposide, and combinations thereof.

Bioactive compounds that can be extracted from plants may include terpenes, terpenoids, flavones and flavonoids, steroids, sterols, saponins and sapogenins, alkanes, alkaloids, amines, amino acids, aldehydes, alcohols, fatty acids, lipids, lignans, phenols, pyrones, butenolides, lactones, chalcones, ketones, benzenes, cyclohexanes, glucosides, glycosides, cyanidins, furans, phorbols, quinones and phloroglucinols. Bioactive compounds that can be extracted from plants also may include large molecular weight materials such as proteins, peptides, enzymes, polysaccharides and carbohydrates.

Plants from which extracts can be prepared and natural substances isolated may include the higher plants: Acanthopanax, Acanthopsis, Acanthosicyos, Acanthus, Achyranthes, Acokanthera, Aconitum, Acorus, Acronychia, Actaea, Actinidia, Adenia, Adhatoda, Aegle, Aesculus, Aframomum, Agastache, Agathosma, Alchemilla, Aleurites, Allium, Aloe, Alonsoa, Aloysia, Alphitonia, Alpinia, Alternanthera, Amaranthus, Amomum, Amphipterygium, Amyris, Anchusa, Ancistrocladus, Anemopsis, Angelica, Annona, Anonidium, Anthemis, Antidesma, Apium, Aralia, Aristolochia, Artemisia, Artocarpus, Asarum, Asclepias, Asimina, Aspalanthus, Asparagus, Aspidosperma, Astragalus, Astronium, Atropa, Avena, Azadirachta, Azara, Baccharis, Bacopa, Balanites, Bambusa, Barleria, Barosma, Bauhinia, Belamcanda, Benincasa, Berberis, Berchemia, Bixa, Bocconia, Borago, Boronia, Boswellia, Brosimum, Brucea, Brunfelsia, Bryonia, Buddleja, Bulnesia, Bupleurum, Bursera, Byrsonima, Calamintha, Calea, Calophyllum, Camellia, Camptotheca, Cananga, Canarium, Canella, Capparis, Capsicum, Carthamus, Carum, Cassia, Cassine, Castanospermum, Catalpa, Catha, Catharanthus, Cayaponia, Cecropia, Centaurea, Centipeda, Centranthus, Cephaelis, Chiranthodendron, Chondrodendron, Chrysophyllum, Cimicifuga, Cinchona, Cinnamomum, Cistus, Citrus, Clausena, Cnicus, Coccoloba, Codonopsis, Coffea, Coix, Cola, Coleus, Colletia, Combreturn, Commiphora, Cordia, Coriaria, Correa, Corydalis, Costus, Crataegus, Croton, Cryptolepis, Cudrania, Cuminum, Cuphea, Cucurma, Cyclanthera, Cymbopogon, Cynara, Cynoglossum, Cyperus, Cyrtocarpa, Dalbergia, Dalea, Danae, Daphne, Datura, Daucus, Decadon, Dendrocalamus, Dendropanax, Deppea, Derris, Desmos, Dichrostachys, Dictamnus, Digitalis, Dillenia, Dioscorea, Dioscoreophyllum, Diosma, Diospyros, Drimys, Duboisia, Duguetia, Dysoxylum, Echinacea, Eclipta, Ehretia, Ekebergia, Eleagnus, Elettaria, Eleutherococcus, Encelia, Entandrophragma, Ephedra, Epimedium, Eriobotrya, Erodium, Eryngium, Erythrochiton, Erythroxylum, Escholzia, Esenbeckia, Euclea, Eucommia, Euodia, Eupatorium, Fabiana, Ferula, Fevillea, Fittonia, Flindersia, Foeniculum, Gallesia, Galphimia, Garcinia, Gaudichaudia, Gaultheria, Gelsemium, Gentiana, Geranium, Gigantochloa, Gingko, Glochidion, Gloeospemum, Grewia, Greyia, Guaiacum, Gymnema, Haematoxylum, Hamamelis, Hamelia, Harpagophytum, Hauya, Heimia, Helleborus, Hieracium, Hierochloe, Hilleria, Hippophae, Houttuynia, Hovenia, Humulus, Huperzia, Hura, Hybanthus, Hydnocarpus, Hydnophytum, Hydrastis, Hydrocotyle, Hymenaea, Hyoscamus, Hypericum, Hyptis, Hyssopus, Iboza, Idiospermum, Ilex, Illicium, Indigofera, Inga, Inula, Iochroma, Iresine, Iris, Jacaranda, Jatropha, Juniperus, Justicia, Kadsura, Kaempferia, Lactuca, Lagochilus, Larrea, Laurus, Lavandula, Lawsonia, Leonurus, Leucas, Ligusticum, Lindera, Lippia, Liriosma, Litsea, Lobelia, Lonchocarpus, Lonicera, Lycium, Macfadyena, Maclura, Mangifera, Mansoa, Marcgravia, Marrubium, Martinella, Matricaria, Maytenus, Medicago, Melissa, Mentha, Mimosa, Mimusops, Mitragyna, Montanoa, Morkillia, Mouriri, Mucuna, Mutisia, Myrica, Myristica, Nardostachys, Nepeta, Nicotiana, Ocotea, Olea, Oncoba, Ophiopogon, Origanum, Pachyrhizus, Panax, Papaver, Pappea, Parthenium, Passiflora, Paullinia, Pelargonium, Penstemon, Perezia, Perilla, Persea, Petiveria, Petroselinum, Peucedanum, Peumus, Pfaffia, Phoebe, Phyllanthus, Phytolacca, Pilocarpus, Pimenta, Pimpinella, Pinellia, Piper, Piqueria, Pithecellobium, Pittosporum, Plectranthus, Pleuropetalum, Podophyllum, Pogostemon, Polygala, Polygonum, Polymnia, Psacalium, Psychotria, Pterygota, Ptychopetalum, Pueraria, Punica, Pycnanthemum, Pygeum, Quararibea, Quassia, Quillaja, Randia, Ratibida, Rauvolfia, Rehmannia, Renealmia, Rheum, Rollinia, Rorippa, Rosmarinus, Rudbeckia, Ruellia, Rumex, Ruscus, Ruta, Saccharum, Salix, Salvia, Sambucus, Sanguinaria, Sapium, Sassafras, Satureja, Sceletium, Schizandra, Securidaca, Securinega, Serenoa, Simmondsia, Smilax, Sophora, Stachytarpheta, Stachys, Staurogyne, Stelechocarpus, Stephania, Sterculia, Stevia, Strophanthus, Strychnos, Symphytum, Syzygium, Tabebuia, Tabemaemontana, Tabemanthe, Tanacetum, Taxus, Tecoma, Terminalia, Teucrium, Thaumatococcus, Tribulus, Trichosanthes, Trifolium, Trigonella, Triplaris, Triumfetta, Tumera, Tussilago, Tylophora, Tynnanthus, Uncaria, Urginea, Urtica, Uvaria, Vaccinium, Valeriana, Vallesia, Vangueria, Vanilla, Vellozia, Vepris, Verbascum, Verbena, Vetiveria, Virola, Viscum, Vismia, Vitex, Voacanga, Warburgia, Withania, Zanthoxylum, Zingiber, Zizyphus and Zygophyllum.

Plant extract compounds may be prepared according methods known to one skilled in the art. For example, the compounds may be purchased from conventional sources, may be readily isolated from specific plants or trees and purified, or may be synthesized using conventional techniques. Therapeutically active plant extract compounds may be modified or derivatized by appending functional groups to enhance specific biological properties. Such modifications are known in the art and may include those that increase the biological penetration into a given biological compartment (e.g. blood, lymphatic system, and central nervous system) and the bioavailability, that enhance the solubility for parenteral administration, and/or that alter the rate of metabolism and excretion.

Pharmaceutically acceptable carriers, adjuvants and vehicles that may be used in the pharmaceutical compositions may include ion exchangers; alumina; aluminum stearate; lecithin; self-emulsifying drug delivery systems (SEDDS) such as alpha-tocopherol polyethyleneglycol 1000 succinate, or other similar polymeric delivery matrices or systems such as nanoparticles; serum proteins such as human serum albumin; buffer substances such as phosphates; glycine; sorbic acid; potassium sorbate; partial glyceride mixtures of saturated vegetable fatty acids; water; salts or electrolytes, such as protamine sulfate, disodium hydrogen phosphate, potassium hydrogen phosphate, sodium chloride, zinc salts; colloidal silica; magnesium trisilicate; polyvinyl pyrrolidone; cellulose-based substances; polyethylene glycol; sodium carboxymethylcellulose; polyacrylates; polyethylene-polyoxypropylene-block polymers; and wool fat.

The pharmaceutical compositions may be administered orally, parenterally, by inhalation spray, topically, rectally, nasally, buccally, vaginally or via an implanted reservoir; however, administration by injection is preferred. The pharmaceutical compositions may contain any conventional non-toxic pharmaceutically acceptable carriers, adjuvants or vehicles. Parenteral administration of the compositions may include subcutaneous, intracutaneous, intravenous, intramuscular, intraperitoneal, intraarticular, intrasynovial, intrasternal, intrathecal, intralesional and intracranial injection or infusion techniques.

The pharmaceutical compositions may be in the form of a sterile injectable preparation, for example, as a sterile injectable suspension. This suspension may be formulated according to techniques known in the art using suitable dispersing agents, surfactants, and suspending agents (e.g. Tween 80). The sterile injectable preparation may also be a sterile injectable solution or suspension in a non-toxic parenterally-acceptable diluent or solvent (e.g. 1,2-propanediol). Acceptable vehicles and solvents may include mannitol, water, Ringers solution and isotonic sodium chloride solution. Furthermore, sterile, fixed oils may be employed as a solvent or a suspending medium. For this purpose, any bland fixed oil may be employed including mono- or diglycerides. Fatty acids, such as oleic acid and its glyceride derivatives, and natural pharmaceutically acceptable oils, such as polyoxyethylated olive oil or castor oil, may be used in the preparation of injectables.

The pharmaceutical composition may have anticancer activity against different types of cancer from the group consisting of non-small cell lung, breast, ovary, prostate, colon, central nervous system (CNS), renal, melanoma, and leukemia in NCI panel of cancer cell lines.

Anticancer activity of the composition against different types of cancer may include lung, breast, and ovary, on the basis of IC50 values and growth inhibition. In one embodiment, a pharmaceutical composition may exhibit anticancer activities for cancer cells selected from the group consisting of lung, breast, ovary, prostate, colon, liver, gastrointestinal, head-and-neck, kidney, neuroblastoma, leukemia, lymphoma, and melanoma. The pharmaceutical composition preferably includes paclitaxel and geranium oil.

In one example, a pharmaceutical composition that includes paclitaxel and geranium oil may inhibit the growth of human lung cancer cells up to 87% at a concentration ranging from 0.5 to 2 μg/ml, with IC50 values ranging from 0.1 to 1.1 μg/ml, which are approximately 18 to 300-fold less than those of paclitaxel alone. The lung cancer cell line may be selected from the group consisting of A-549, NCI-H1437 and NCI-H838.

In another example, a pharmaceutical composition that includes paclitaxel and geranium oil may inhibit the growth of human breast cancer cells up to 87% at a concentration ranging from 0.5 to 2 μg/ml, with IC50 values ranging from 1.1 to 1.4 μg/ml, which are approximately 23 to 42-fold less than those of paclitaxel alone. The breast cancer cell line may be selected from the group consisting of MCF-7 and MDA-MB-231.

In a further example, a pharmaceutical composition that includes paclitaxel and geranium oil may inhibit the growth of human ovarian cancer cells up to 93% at a concentration ranging from 0.5 to 2 μg/ml, with an IC50 value of 1.1 μg/ml, which is approximately 51-fold less than that of paclitaxel alone. The ovarian cancer cell line may be, for example, SK-OV-3.

In these examples, the pharmaceutical composition may be administered to the patient in combination with a pharmaceutically acceptable additive, carrier, diluent, solvent, filter, lubricant, excipient, binder, or stabilizer. Moreover, the pharmaceutical composition may be administered systemically, orally, and/or by any other suitable method.

In another embodiment, a pharmaceutical composition may exhibit anticancer activities for cancer cells selected from the group consisting of lung, breast, ovary, prostate, colon, liver, gastrointestinal, head-and-neck, kidney, neuroblastoma, leukemia, lymphoma, and melanoma. The pharmaceutical composition may include paclitaxel and geraniol. The cancer cell lines used may be selected from the group consisting of lung cell A-549, NCI-H1437 and NCI-H838, breast cell MCF-7 and MDA-MB-231, and ovarian cell SK-OV-3.

In one example, a pharmaceutical composition that includes paclitaxel and geraniol may inhibit the growth of lung cancer cells up to 91% at a concentration ranging from 0.125 to 2 μg/ml, with IC50 values ranging from less than 0.125 to 1.1 μg/ml, which are approximately 18 to 3000-fold less than those of paclitaxel alone.

In another example, a pharmaceutical composition that includes paclitaxel and geraniol may inhibit the growth of breast cancer cells up to 87% at a concentration ranging from 0.5 to 2 μg/ml, with IC50 values ranging from 0.7 to 0.8 μg/ml, which are approximately 41 to 71-fold less than those of paclitaxel alone.

In a further example, a pharmaceutical composition that includes paclitaxel and geraniol may inhibit the growth of ovarian cancer cells up to 87% at a concentration ranging from 0.5 to 2 μg/ml, with an IC50 value of 1.1 μg/ml, which is approximately 51-fold less than that of paclitaxel alone.

In these examples, the pharmaceutical composition may be administered to the patient in combination with a pharmaceutically acceptable additive, carrier, diluent, solvent, filter, lubricant, excipient, binder, or stabilizer. Moreover, the pharmaceutical composition may be administered systemically, orally, and/or by any other suitable method.

In a further embodiment, a pharmaceutical composition may exhibit anticancer activities for cancer cells selected from the group consisting of lung, breast, ovary, prostate, colon, liver, gastrointestinal, head-and-neck, kidney, neuroblastoma, leukemia, lymphoma, and melanoma. The pharmaceutical composition may include fluorouracil (5-FU) and beta-citronellol. The cancer cell lines used may be selected from the group consisting of lung cell A-549, NCI-H1437 and NCI-H838, breast cell MCF-7 and MDA-MB-231, and ovarian cell SK-OV-3.

In one example, a pharmaceutical composition that includes 5-FU and beta-citronellol may inhibit the growth of lung cancer cells up to 71% at a concentration ranging from 6.25 to 25 μg/ml, with IC50 values ranging from less than 4.0 to 25 μg/ml, which are approximately 1.5-fold less than those of 5-FU alone.

In another example, a pharmaceutical composition that includes 5-FU and beta-citronellol may inhibit the growth of breast cancer cells up to 88% at a concentration ranging from 6.25 to 25 μg/ml, with IC50 values ranging from 4.2 to 6.3 μg/ml, which are approximately 9 to 50-fold less than those of 5-FU alone.

In a further example, a pharmaceutical composition that includes 5-FU and beta-citronellol may inhibit the growth of ovarian cancer cells up to 90% at a concentration ranging from 6.25 to 25 μg/ml, with an IC50 value of 9.5 μg/ml, which is approximately 7-fold less than that of 5-FU alone.

In these examples, the pharmaceutical composition may be administered to the patient in combination with a pharmaceutically acceptable additive, carrier, diluent, solvent, filter, lubricant, excipient, binder, or stabilizer. Moreover, the pharmaceutical composition may be administered systemically, orally, and/or by any other suitable method.

In another embodiment, a method of treating a patient with cancers related to the lung, breast, ovary, prostate, colon, liver, gastrointestinal, head-and-neck, kidney, neuroblastoma, leukemia, lymphoma, and melanoma includes administering a pharmaceutical composition containing paclitaxel and geranium oil to the patient. The composition may inhibit the growth of human cancer cells consisting of lung cells, breast cells and ovarian cells up to 87%, 87% and 93%, respectively.

The cancer cell lines may be selected from the group consisting of lung cell A-549, NCI-H1437 and NCI-H838, breast cell MCF-7 and MDA-MB-231, and ovarian cell SK-OV-3. The composition may be used singly or in combination with a pharmaceutically acceptable carrier. The composition may be administered to the patient in combination with a pharmaceutically acceptable additive, carrier, diluent, solvent, filter, lubricant, excipient, binder, or stabilizer. The composition may be administered systemically, orally, and/or by any other suitable method.

In a further embodiment, a method of treating a patient with cancers related to the lung, breast, ovary, prostate, colon, liver, gastrointestinal, head-and-neck, kidney, neuroblastoma, leukemia, lymphoma, and melanoma includes administering a pharmaceutical composition containing paclitaxel and geraniol to the patient. The composition may inhibit the growth of human cancer cells consisting of lung cells, breast cells and ovarian cells up to 91%, 87% and 87%, respectively.

The cancer cell lines may be selected from the group consisting of lung cell A-549, NCI-H1437 and NCI-H838, breast cell MCF-7 and MDA-MB-231, and ovarian cell SK-OV-3. The composition may be used singly or in combination with a pharmaceutically acceptable carrier. The composition may be administered to the patient in combination with a pharmaceutically acceptable additive, carrier, diluent, solvent, filter, lubricant, excipient, binder, or stabilizer. The composition may be administered systemically, orally, and/or by any other suitable method.

In another embodiment, a method of treating a patient with cancers related to lung, breast, ovary, prostate, colon, liver, gastrointestinal, head-and-neck, kidney, neuroblastoma, leukemia, lymphoma, and melanoma includes administering a pharmaceutical composition containing fluorouracil (5-FU) and beta-citronellol to the patient. The composition may inhibit the growth of human cancer cells consisting of lung cells, breast cells and ovarian cells up to 71%, 88% and 90%, respectively.

The cancer cell lines may be selected from the group consisting of lung cell A-549, NCI-H1437 and NCI-H838, breast cell MCF-7 and MDA-MB-231, and ovarian cell SK-OV-3. The composition may be used singly or in combination with a pharmaceutically acceptable carrier. The composition may be administered to the patient in combination with a pharmaceutically acceptable additive, carrier, diluent, solvent, filter, lubricant, excipient, binder, or stabilizer. The composition may be administered systemically, orally, and/or by any other suitable method.

In a further embodiment, the combination of paclitaxel and geraniol may exhibit in vivo antitumor efficacy against human breast tumor xenograft implanted in athymic mice. The breast tumor xenograft implanted in athymic mice may be, for example, MDA-MB-231.

The composition may inhibit the tumor growth in MDA-MB-231 breast tumor-bearing athymic mice in a similar manner as compared to the paclitaxel alone, and has the advantage of using 25-fold less amount of paclitaxel in the composition.

The compositions and methods of the present invention will be further illustrated in the following non-limiting Examples. The Examples are illustrative of various embodiments only and do not limit the claimed invention regarding the materials, conditions, weight ratios, process parameters and the like recited herein.

### EXAMPLE 1

### Cancer Screening Using Cell Panel

Anti-cancer agents were screened using a screening system provided and performed by the National Cancer Institute (NCI) (http://dtp.nci.nih.gov/docs/misc/common_files/submit_compounds.html). The Developmental Therapeutics Program (DTP) of NCI operates an anti-cancer screening program. The program accepts both natural and synthetic compounds. For screening of natural products extracts, the Natural Products Branch can be contacted.

DTP Human Tumor Cell Line Screen Process: The operation of this screen utilizes 60 different human tumor cell lines, representing leukemia, melanoma and cancers of the lung, colon, brain, ovary, breast, prostate, and kidney. The aim is to prioritize for further evaluation synthetic compounds or natural product samples showing selective growth inhibition or cell killing of particular tumor cell lines. This screen is unique in that the complexity of a 60 cell line dose response produced by a given compound results in a biological response pattern, which can be utilized in pattern recognition algorithms. Using these algorithms, it is possible to assign a putative mechanism of action to a test compound, or to determine that the response pattern is unique and not similar to that of any of the standard prototype compounds included in the NCI database. In addition, following characterization of various cellular molecular targets in the 60 cell lines, it may be possible to select compounds most likely to interact with a specific molecular target.

Methodology of the In Vitro Cancer Screen: The human tumor cell lines of the cancer screening panel are grown in RPMI 1640 medium containing 5% fetal bovine serum and 2 mM L-glutamine. For a typical screening experiment, cells are inoculated into 96 well microtiter plates in 100 μl at plating densities ranging from 5,000 to 40,000 cells/well depending on the doubling time of individual cell lines. After cell inoculation, the microtiter plates are incubated at 37° C., 5% CO₂, 90% relative humidity for 24 hours prior to addition of experimental drugs.

After 24 hours, two plates of each cell line are fixed in situ with TCA, to represent a measurement of the cell population for each cell line at the time of drug addition (Tz). Experimental drugs are solubilized in dimethyl sulfoxide at 400-fold the desired final maximum test concentration and stored frozen prior to use. At the time of drug addition, an aliquot of frozen concentrate is thawed and diluted to twice the desired final maximum test concentration with complete medium containing 50 μg/ml gentamicin. Four additional dilutions, i.e. 10-fold or ½ log serial dilutions, are made to provide a total of five drug concentrations, plus a control. Aliquots of 100 μl of these different drug dilutions are added to the appropriate microtiter wells already containing 100 μl of medium, resulting in the required final drug concentrations.

Following drug addition, the plates are incubated for an additional 48 hours at 37° C., 5% CO₂, 90% relative humidity. For adherent cells, the assay is terminated by the addition of cold TCA. Cells are fixed in situ by the gentle addition of 50 μl of cold 50% (w/v) TCA (final concentration, 10% TCA) and incubated for 60 minutes at 4° C. The supernatant is discarded, and the plates are washed five times with tap water and air dried. Sulforhodamine B (SRB) solution (100 μl) at 0.4% (w/v) in 1% acetic acid is added to each well, and plates are incubated for 10 minutes at room temperature. After staining, unbound dye is removed by washing five times with 1% acetic acid, and the plates are air dried. Bound stain is subsequently solubilized with 10 mM trizma base, and the absorbance is read on an automated plate reader at a wavelength of 515 nm. For suspension cells, the methodology is the same except that the assay is terminated by fixing settled cells at the bottom of the wells by gently adding 50 μl of 80% TCA (final concentration, 16% TCA). Using the seven absorbance measurements [time zero, (Tz), control growth, (C), and test growth in the presence of drug at the five concentration levels (Ti)], the percentage growth is calculated at each of the drug concentrations levels. Percentage growth inhibition is calculated as:

[(Ti−Tz)/(C−Tz)]×100 for concentrations for which Ti≧Tz

[(Ti−Tz)/Tz]×100 for concentrations for which Ti<Tz

Three dose response parameters are calculated for each experimental agent. Growth inhibition of 50% (GI50) is calculated from [(Ti−Tz)/(C−Tz)]×100=50, which is the drug concentration resulting in a 50% reduction in the net protein increase (as measured by SRB staining) in control cells during the drug incubation. The drug concentration resulting in total growth inhibition (TGI) is calculated from Ti=Tz. The LC50 (concentration of drug resulting in a 50% reduction in the measured protein at the end of the drug treatment as compared to that at the beginning) indicating a net loss of cells following treatment is calculated from [(Ti−Tz)/Tz]×100=−50. Values are calculated for each of these three parameters if the level of activity is reached; however, if the effect is not reached or is exceeded, the value for that parameter is expressed as greater or less than the maximum or minimum concentration tested.

Results of screening for a paclitaxel-geraniol composition using human cancer cell lines from NCI are shown in TABLE 1. The paclitaxel-geraniol composition demonstrated significant anticancer activity against lung (non-small cell), breast, colon, CNS, melanoma, ovarian, renal, and prostate cancers from the NCI database.

TABLE 1
GI50, TGI and LC50 values (μg/ml) of paclitaxel-geraniol composition

| Panel/Cell line | GI50 | TGI | LC50 |
|---|---|---|---|
| Lung Cancer (non-small cell) | | | |
| A549/ATCC | 1.66E−5 | 10.95 | >12.5 |
| EKVX | 30.97 | >12.5 | >12.5 |
| HOP-62 | 2.11E−2 | >12.5 | >12.5 |
| NCI-H23 | 0.37 | >12.5 | >12.5 |
| NCI-H226 | 53.69 | >12.5 | >12.5 |
| NCI-H322M | 4.59E−6 | 8.18 | >12.5 |
| NCI-H460 | 3.82E−3 | 7.25 | >12.5 |
| NCI-H522 | 6.54E−4 | >12.5 | >12.5 |
| Breast cancer | | | |
| MCF-7 | 2.91E−2 | >12.5 | >12.5 |
| NCI/ADR-RES | 0.58 | 6.49 | >12.5 |
| HS-578T | 7.61E−3 | 9.19 | >12.5 |
| MDA-MB-231 | 5.39E−3 | 0.35 | >12.5 |
| MDA-MB-435 | 3.46E−4 | 5.65E−3 | 0.35 |
| BT-549 | 5.51E−7 | 3.69E−6 | 3.70 |
| T-47D | 6.67E−3 | >12.5 | >12.5 |
| Colon cancer | | | |
| COLO-205 | <1.00E−6 | 3.50E−3 | 3.60E−2 |
| HCT-116 | <1.00E−6 | <4.50E−3 | 5.46E−3 |
| KM-12 | <1.00E−6 | 3.6E−3 | 0.19 |
| SW-620 | <1.00E−6 | <4.50E−3 | 2.97E−2 |
| CNS cancer | | | |
| SF-295 | 1.19E−3 | 1.30E−2 | 2.40E−2 |
| SF-539 | 1.09E−3 | 3.80E−3 | 1.40E−2 |
| SNB-75 | 9.20E−4 | 1.36E−2 | 2.90E−2 |
| Melanoma | | | |
| M14 | 1.00E−6 | 1.40E−3 | 0.11 |
| MALME-3M | 1.10E−3 | 9.30E−3 | 7.70E−2 |
| SK-MEL-2 | 1.19E−3 | 3.48E−3 | 1.02E−2 |
| SK-MEL-28 | <1.00E−6 | <1.00E−6 | 8.67E−3 |
| Ovarian cancer | | | |
| IGR-OV1 | <1.00E−6 | <1.00E−6 | 0.51 |
| OVCAR-3 | <1.00E−6 | 1.64E−3 | 1.65E−2 |
| OVCAR-4 | <1.00E−6 | 4.50E−3 | 0.18 |
| SK-OV3 | <1.00E−6 | 1.38 | >45.0 |
| Renal cancer | | | |
| A498 | 1.07E−3 | 8.20E−3 | >45.0 |
| CAKI-1 | 5.53E−3 | >1.00E−2 | >1.00E−2 |
| Prostate cancer | | | |
| DU-145 | <1.00E−6 | <1.00E−6 | 5.00E−3 |
| PC-3 | <1.00E−6 | <1.00E−6 | <1.00E−6 |

### EXAMPLE 2

### In Vitro Cytotoxicity of a Pharmaceutical Composition Containing Paclitaxel and Geranium Oil Against Human Cancer Cell Lines

The human cancer cell lines were commercially obtained from American Type Culture Collection (ATCC), Manassas, Va., USA. Cells were grown in tissue culture flasks in complete growth medium (RPMI-1640 medium with 2 mM glutamine, pH 7.4, sterilized by filtration and supplemented with 10% sterilized fetal calf serum and 100 units/ml penicillin-streptomycin before use) at 37° C. in an atmosphere of 5% CO₂ and 90% relative humidity in a carbon dioxide incubator. The cells at sub-confluent stage were harvested from the flask by treatment with trypsin (0.05% trypsin in PBS containing 0.02% EDTA) and suspended in complete growth medium. Cells with cell viability of more than 97% by trypan blue exclusion technique were used for determination of cytotoxicity.

The suspension of human cancer cell lines of required cell density (5000-10000 cells) in complete growth medium was prepared and cell suspension (100 μl per well for each cell line) was added into a 96-well tissue culture plate. Four additional wells of each cell line were also prepared for control. The plates were incubated overnight at 37° C. in an atmosphere of 5% CO₂ and 90% relative humidity in a CO₂ incubator.

Paclitaxel was dissolved in geranium oil and ethanol to obtain a stock solution of 10 mg/ml, which was then serially diluted with complete growth medium to obtain three working test solutions of 2, 0.5 and 0.125 μg/ml for paclitaxel in the combination. The working solutions of paclitaxel-geranium oil composition at different concentrations (100 μl per well) were added after 24-hour incubation of cells in the designated wells. Equivalent amount of complete growth medium was added as control. The plates were further incubated for 48 hours at 37° C. in an atmosphere of 5% CO₂ and 90% relative humidity in a CO₂ incubator. The number of metabolically viable cells was determined by MTT tetrazolium dye assay.

Subsequently, 5 mg/ml of MTT (20 μl per well) was added per well, and cells were then further incubated for 3 hours. The medium was removed, and the blue black MTT formazan produced by metabolically active cells was dissolved with 100 μl per well of dimethyl sulphoxide (DMSO). Absorbance was determined at 550 nm using an ELISA plate reader. The percent cell growth in presence of test material was determined considering the cell growth in absence of test material (control) as 100%, and in turn percent inhibition was calculated. Cytotoxicity was also measured as the IC50, the concentration of test agent that produces 50% reduction in the number of viable cells (MTT absorbance) when compared to untreated controls.

In vitro cytotoxicity of paclitaxel-geranium oil composition was determined against human lung (A-549, NCI-H1437, and NCI-H838), breast (MCF-7 and MDA-MB-231), and ovarian (SK-OV-3) cancer cell lines. The results are summarized in TABLE 2. Paclitaxel-geranium oil composition showed dose-dependent inhibition of cell growth of the human cancer cell lines studied. The maximum growth inhibition varied from 58 to 93% at 2 μg/ml. The present composition was most effective against human lung cancer cell lines A-549 and NCI-H838, and markedly inhibited the growth of breast cancer cell line MCF-7 and ovarian cancer cell line SK-OV-3. When compared to paclitaxel alone, the present composition was much more efficacious towards all the six tested cell lines, as evidenced by about 18- to 300-fold less IC50 values.

TABLE 2
In vitro cytotoxicity of Paclitaxel-Geranium Oil
Composition against human cancer cell lines

| | | Growth | | | | |
|---|---|---|---|---|---|---|
| | | inhibition (%) | | | | |
| | | Composition | | | | |
| | | (μg/ml) | | | IC50 (μg/ml) | |
| | | 0.125 | 0.5 | 2 | Composition | Paclitaxel |
| Lung | A-549 | 29 | 39 | 87 | 1.1 | 20.1 |
| Lung | NCI-H1437 | 36 | 47 | 58 | 0.9 | 36.8 |
| Lung | NCI-H838 | 52 | 61 | 85 | 0.1 | 30.9 |
| Breast | MCF-7 | 20 | 31 | 87 | 1.1 | 46.0 |
| Breast | MDA-MB-231 | 24 | 40 | 65 | 1.4 | 31.9 |
| Ovary | SK-OV-3 | 9 | 29 | 93 | 1.1 | 56.5 |

### EXAMPLE 3

### In Vitro Cytotoxicity of a Pharmaceutical Composition Containing Paclitaxel and Geraniol against Human Cancer Cell Lines

Human cancer cell lines were grown and harvested, and cytotoxicity was determined exactly as per Example 2, except that the test material used was geraniol, instead of geranium oil, and three working test solutions were prepared of the same concentrations as in Example 2.

In vitro cytotoxicity of paclitaxel-geraniol composition was determined against human lung (A-549, NCI-H1437, and NCI-H838), breast (MCF-7 and MDA-MB-231), and ovarian (SK-OV-3) cancer cell lines. The results are summarized in TABLE 3. Paclitaxel-geraniol composition showed dose-dependent inhibition of cell growth of the human cancer cell lines studied. The maximum growth inhibition varied from 85 to 91% at 2 μg/ml. As evidenced by the IC50 values, the present composition was most effective against human lung cancer cell lines NCI-H1437 and NCI-H838. It was also prominently cytotoxic against human breast cancer cell lines MCF-7 and MDA-MB-231. When compared to paclitaxel alone, the present composition was much more efficacious towards all the tested cell lines, as evidenced by about 18- to 3000-fold less IC50 values.

TABLE 3
In vitro cytotoxicity of Paclitaxel-Geraniol Composition
against human cancer cell lines

| | | Growth | | | | |
|---|---|---|---|---|---|---|
| | | inhibition (%) | | | | |
| | | Composition | | | | |
| | | (μg/ml) | | | IC50 (μg/ml) | |
| | | 0.125 | 0.5 | 2 | Composition | Paclitaxel |
| Lung | A-549 | 43 | 42 | 85 | 1.1 | 20.1 |
| Lung | NCI-H1437 | 67 | 71 | 90 | 0.01 | 36.8 |
| Lung | NCI-H838 | 74 | 72 | 91 | 0.01 | 30.9 |
| Breast | MCF-7 | 44 | 44 | 86 | 0.7 | 46.0 |
| Breast | MDA-MB-231 | 42 | 46 | 87 | 0.8 | 31.9 |
| Ovary | SK-OV-3 | 27 | 32 | 87 | 1.1 | 56.5 |

### EXAMPLE 4

### In Vitro Cytotoxicity of a Pharmaceutical Composition Containing Fluorouracil and Beta-citronellol against Human Cancer Cell Lines

Human cancer cell lines were grown and harvested, and cytotoxicity was determined exactly as per Example 2, except that the test materials used were 5-FU and beta-citronellol, and three working test solutions were prepared at 25, 6.25, 1.56 μg/ml.

In vitro cytotoxicity of 5-FU-beta-citronellol composition was determined against human lung (A-549, NCI-H1437, and NCI-H838), breast (MCF-7 and MDA-MB-231), and ovarian (SK-OV-3) cancer cell lines. The results are summarized in TABLE 4. 5-FU-beta-citronellol composition showed dose-dependent inhibition of cell growth of the human cancer cell lines studied. The inhibition varied from 45 to 90% at 25 μg/ml. Based on both percent growth inhibition and IC50 values, the present composition was most effective against human breast cancer cell line MCF-7, and exerted considerable extent of growth inhibition towards breast (MDA-MB-231), ovarian (SK-OV-3), and lung cancer cell lines (A-549 and NCI-H838). When compared to 5-FU alone, the present composition was much more efficacious towards all the six tested cell lines, as evidenced by about 1.5- to 50-fold less IC50 values.

TABLE 4
In vitro cytotoxicity of 5-FU-Beta-citronellol Composition
against human cancer cell lines

| | | Growth inhibition (%) | | | | |
|---|---|---|---|---|---|---|
| | | Composition (μg/ml) | | | IC50 (μg/ml) | |
| | | 0.125 | 0.5 | 2 | Composition | 5-FU |
| Lung | A-549 | 49 | 58 | 70 | 4.0 | 5.9 |
| Lung | NCI-H1437 | 40 | 40 | 45 | >25 | >100 |
| Lung | NCI-H838 | 40 | 49 | 71 | 7.5 | 12.1 |
| Breast | MCF-7 | 33 | 63 | 88 | 4.2 | 38.7 |
| Breast | MDA-MB-231 | 18 | 50 | 88 | 6.3 | 327.9 |
| Ovary | SK-OV-3 | 21 | 41 | 90 | 9.5 | 66.6 |

### EXAMPLE 5

### In Vivo Antitumor Efficacy Test of Composition Comprising Paclitaxel and Geraniol

The antitumor efficacy of composition comprising paclitaxel and geraniol against the subcutaneously implanted solid tumors induced by MDA-MB-231 cells in nude mice was evaluated. Treatments were initiated when the tumors inoculated in nude mice reached a tumor volume of 50-100 mm³, and this day was designated as Day 1. Mice were divided into 3 different groups consisting of five mice in each group, and administered intraperitoneally with normal saline (control), paclitaxel (5 mg/kg) in normal saline, or composition comprising paclitaxel (0.2 mg/kg) and geraniol (45 mg/kg) twice a week for 28 days. The size of tumor and change of body weight of each mouse were recorded.

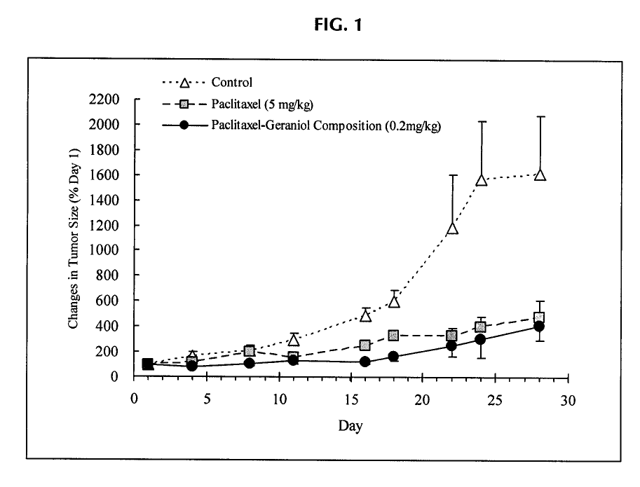

The anticancer efficacy of the paclitaxel-geraniol composition was studied in breast tumor-bearing nude mice. FIG. 1 illustrates the progress of tumor growth observed for 28 days in nude mice injected with normal saline (control), paclitaxel, or paclitaxel-geraniol composition. In the control, mice were injected with normal saline. In Paclitaxel, mice were injected with paclitaxel (5 mg/kg) in normal saline. In the Paclitaxel-geraniol composition, mice were injected with composition comprising paclitaxel (0.2 mg/kg) and geraniol in normal saline.

For the control, the size of tumor was found to increase significantly with time, indicating that normal saline had no significant effect in inhibiting tumor growth. In contrast, the group injected with paclitaxel or paclitaxel-geraniol composition significantly suppressed the tumor growth in a similar manner, as compared to the control group (p<0.001, repeated measures ANOVA). The results demonstrated that 25-fold less amount of paclitaxel (0.2 mg/kg) in the composition advantageously exhibited similar inhibitory effect on breast tumor growth in vivo when compared with paclitaxel (5 mg/kg) alone.

While there have been described and pointed out fundamental novel features of the invention as applied to a preferred embodiment thereof, it will be understood that various omissions and substitutions and changes, in the form and details of the embodiments illustrated, may be made by those skilled in the art without departing from the spirit of the invention. The invention is not limited by the embodiments described above, which are presented as examples only but can be modified in various ways within the scope of protection defined by the appended patent claims.

What is claimed is:

1. A cancer treatment composition, comprising:
geranium oil; and
a chemotherapeutic agent or plant extract selected from the group consisting of plant-derived bioactive compounds,
wherein said cancer is selected from the group consisting of lung, breast, ovary, prostate, colon, liver, gastrointestinal, head-and-neck, cervix, kidney, neuroblastoma, leukemia, lymphoma, and melanoma.

2. The composition of claim 1, further comprising at least one additional component of geranium oil.

3. The composition of claim 1, wherein said geranium oil is extracted from one or more species of the genus Pelargonium.

4. The composition of claim 3, wherein said geranium oil is further extracted from a plant of the species graveolens.

5. The composition of claim 3, wherein said geranium oil is further extracted from a plant of the species capitatum.

6. The composition of claim 3, wherein said geranium oil is further extracted from a plant of the species roseum.

7. The composition of claim 1, wherein said chemotherapeutic agent is selected from the group consisting of cyclophosphamide, chlorambucil, melphalan, mechlorethamine, ifosfamide, busulfan, lomustine, streptozocin, temozolomide, dacarbazine, cisplatin, carboplatin, oxaliplatin, procarbazine, uramustine, methotrxate, pemetrexed, fludarabine, cytarabine, fluorouracil, floxuridine, gemcitabine, capecitabine, vinblastine, vincristine, vinorelbine, etoposide, paclitaxel, docetaxel, doxorubicin, daunorubicin, epirubicin, idarubicin, mitoxantrone, bleomycin, mitomycin, hydroxyurea, topotecan, irinotecan, amsacrine, teniposide, and combinations thereof.

8. The composition of claim 1, wherein said geranium oil is selected from the group consisting of citronellol, geraniol, geranyl formate, and citronellyl formate, linalool, eugenol, myrtenol, terpineol, citral, methone, sabinene, and combinations thereof.

9. The composition of claim 8, wherein said chemotherapeutic agent is selected from the group consisting of cyclophosphamide, chlorambucil, melphalan, mechlorethamine, ifosfamide, busulfan, lomustine, streptozocin, temozolomide, dacarbazine, cisplatin, carboplatin, oxaliplatin, procarbazine, uramustine, methotrxate, pemetrexed, fludarabine, cytarabine, fluorouracil, floxuridine, gemcitabine, capecitabine, vinblastine, vincristine, vinorelbine, etoposide, paclitaxel, docetaxel, doxorubicin, daunorubicin, epirubicin, idarubicin, mitoxantrone, bleomycin, mitomycin, hydroxyurea, topotecan, irinotecan, amsacrine, teniposide, and combinations thereof.

10. The composition of claim 1, further comprising a plant extract selected from the genus consisting of Acanthopanax, Acanthopsis, Acanthosicyos, Acanthus, Achyranthes, Acokanthera, Aconitum, Acorus, Acronychia, Actaea, Actinidia, Adenia, Adhatoda, Aegle, Aesculus, Aframomum, Agastache, Agathosma, Alchemilla, Aleurites, Allium, Aloe, Alonsoa, Aloysia, Alphitonia, Alpinia, Alternanthera, Amaranthus, Amomum, Amphipterygium, Amyris, Anchusa, Ancistrocladus, Anemopsis, Angelica, Annona, Anonidium, Anthemis, Antidesma, Apium, Aralia, Aristolochia, Artemisia, Artocarpus, Asarum, Asclepias, Asimina, Aspalanthus, Asparagus, Aspidosperma, Astragalus, Astronium, Atropa, Avena, Azadirachta, Azara, Baccharis, Bacopa, Balanites, Bambusa, Barleria, Barosma, Bauhinia, Belamcanda, Benincasa, Berberis, Berchemia, Bixa, Bocconia, Borago, Boronia, Boswellia, Brosimum, Brucea, Brunfelsia, Bryonia, Buddleja, Bulnesia, Bupleurum, Bursera, Byrsonima, Calamintha, Calea, Calophyllum, Camellia, Camptotheca, Cananga, Canarium, Canella, Capparis, Capsicum, Carthamus, Carum, Cassia, Cassine, Castanospermum, Catalpa, Catha, Catharanthus, Cayaponia, Cecropia, Centaurea, Centipeda, Centranthus, Cephaelis, Chiranthodendron, Chondrodendron, Chrysophyllum, Cimicifuga, Cinchona, Cinnamomum, Cistus, Citrus, Clausena, Cnicus, Coccoloba, Codonopsis, Coffea, Coix, Cola, Coleus, Colletia, Combreturn, Commiphora, Cordia, Coriaria, Correa, Corydalis, Costus, Crataegus, Croton, Cryptolepis, Cudrania, Cuminum, Cuphea, Cucurma, Cyclanthera, Cymbopogon, Cynara, Cynoglossum, Cyperus, Cyrtocarpa, Dalbergia, Dalea, Danae, Daphne, Datura, Daucus, Decadon, Dendrocalamus, Dendropanax, Deppea, Derris, Desmos, Dichrostachys, Dictamnus, Digitalis, Dillenia, Dioscorea, Dioscoreophyllum, Diosma, Diospyros, Drimys, Duboisia, Duguetia, Dysoxylum, Echinacea, Eclipta, Ehretia, Ekebergia, Eleagnus, Elettaria, Eleutherococcus, Encelia, Entandrophragma, Ephedra, Epimedium, Eriobotrya, Erodium, Eryngium, Erythrochiton, Erythroxylum, Escholzia, Esenbeckia, Euclea, Eucommia, Euodia, Eupatorium, Fabiana, Ferula, Fevillea, Fittonia, Flindersia, Foeniculum, Gallesia, Galphimia, Garcinia, Gaudichaudia, Gaultheria, Gelsemium, Gentiana, Geranium, Gigantochloa, Gingko, Glochidion, Gloeospemum, Grewia, Greyia, Guaiacum, Gymnema, Haematoxylum, Hamamelis, Hamelia, Harpagophytum, Hauya, Heimia, Helleborus, Hieracium, Hierochloe, Hilleria, Hippophae, Houttuynia, Hovenia, Humulus, Huperzia, Hura, Hybanthus, Hydnocarpus, Hydnophytum, Hydrastis, Hydrocotyle, Hymenaea, Hyoscamus, Hypericum, Hyptis, Hyssopus, Iboza, Idiospermum, Ilex, Illicium, Indigofera, Inga, Inula, Iochroma, Iresine, Iris, Jacaranda, Jatropha, Juniperus, Justicia, Kadsura, Kaempferia, Lactuca, Lagochilus, Larrea, Laurus, Lavandula, Lawsonia, Leonurus, Leucas, Ligusticum, Lindera, Lippia, Liriosma, Litsea, Lobelia, Lonchocarpus, Lonicera, Lycium, Macfadyena, Maclura, Mangifera, Mansoa, Marcgravia, Marrubium, Martinella, Matricaria, Maytenus, Medicago, Melissa, Mentha, Mimosa, Mimusops, Mitragyna, Montanoa, Morkillia, Mouriri, Mucuna, Mutisia, Myrica, Myristica, Nardostachys, Nepeta, Nicotiana, Ocotea, Olea, Oncoba, Ophiopogon, Origanum, Pachyrhizus, Panax, Papaver, Pappea, Parthenium, Passiflora, Paullinia, Pelargonium, Penstemon, Perezia, Perilla, Persea, Petiveria, Petroselinum, Peucedanum, Peumus, Pfaffia, Phoebe, Phyllanthus, Phytolacca, Pilocarpus, Pimenta, Pimpinella, Pinellia, Piper, Piqueria, Pithecellobium, Pittosporum, Plectranthus, Pleuropetalum, Podophyllum, Pogostemon, Polygala, Polygonum, Polymnia, Psacalium, Psychotria, Pterygota, Ptychopetalum, Pueraria, Punica, Pycnanthemum, Pygeum, Quararibea, Quassia, Quillaja, Randia, Ratibida, Rauvolfia, Rehmannia, Renealmia, Rheum, Rollinia, Rorippa, Rosmarinus, Rudbeckia, Ruellia, Rumex, Ruscus, Ruta, Saccharum, Salix, Salvia, Sambucus, Sanguinaria, Sapium, Sassafras, Satureja, Sceletium, Schizandra, Securidaca, Securinega, Serenoa, Simmondsia, Smilax, Sophora, Stachytarpheta, Stachys, Staurogyne, Stelechocarpus, Stephania, Sterculia, Stevia, Strophanthus, Strychnos, Symphytum, Syzygium, Tabebuia, Tabernaemontana, Tabernanthe, Tanacetum, Taxus, Tecoma, Terminalia, Teucrium, Thaumatococcus, Tribulus, Trichosanthes, Trifolium, Trigonella, Triplaris, Triumfetta, Tumera, Tussilago, Tylophora, Tynnanthus, Uncaria, Urginea, Urtica, Uvaria, Vaccinium, Valeriana, Vallesia, Vangueria, Vanilla, Vellozia, Vepris, Verbascum, Verbena, Vetiveria, Virola, Viscum, Vismia, Vitex, Voacanga, Warburgia, Withania, Zanthoxylum, Zingiber, Zizyphus, Zygophyllum, and combinations thereof.

11. The composition of claim 1, further comprising a bioactive compound extracted from plants, the bioactive compound selected from the group consisting of terpenes, terpenoids, flavones and flavonoids, steroids, sterols, saponins and sapogenins, alkanes, alkaloids, amines, amino acids, aldehydes, alcohols, fatty acids, lipids, lignans, phenols, pyrones, butenolides, lactones, chalcones, ketones, benzenes, cyclohexanes, glucosides, glycosides, cyanidins, furans, phorbols, quinones and phloroglucinols.

12. The composition of claim 1, further comprising a bioactive compound that is a large molecular weight material isolated from plants, the bioactive compound selected from the group consisting of protein, peptide, enzyme, polysaccharide and carbohydrate.

13. The composition of claim 1, wherein said cancer is selected from the group consisting of non-small cell lung, breast, ovary, prostate, colon, central nervous system (CNS), renal, melanoma, and leukemia, in NCI panel of cancer cell lines.

14. The composition of claim 1, wherein said cancer is from a lung cancer cell line selected from the group consisting of A-549, NCI-H838, and NCI-H1437; and wherein said composition is configured to inhibit the growth of lung cancer cells up to 91%.

15. The composition of claim 14, wherein said composition has a concentration from 0.5 to 25 μg/ml.

16. The composition of claim 1, wherein said cancer is from a breast cancer cell line selected from the group consisting of MCF-7 and MDA-MB-231, and wherein said composition is configured to inhibit the growth of breast cancer cells up to 88%.

17. The composition of claim 16, wherein said composition has a concentration from 0.5 to 25 μg/ml.

18. The composition of claim 1, wherein said cancer is from an ovarian cancer cell line selected from SK-OV-3, and wherein said composition is configured to inhibit the growth of ovarian cancer cells up to 93%.

19. The composition of claim 18, wherein said composition has a concentration from 0.5 to 25 μg/ml.

20. The composition of claim 1, wherein said composition is configured to be administered to a patient with a pharmaceutically acceptable carrier selected from the group consisting of an additive, a diluent, a solvent, a filter, a lubricant, an excipient, a binder, a stabilizer and combinations thereof.

21. The composition as claimed in claim 1, wherein said composition can be administered systemically and/or orally.

22. A method of treating cancer, comprising: administering a cancer treatment composition, wherein said cancer treatment composition comprises geranium oil and a chemotherapeutic agent or plant extract selected from the group consisting of plant-derived bioactive compounds.

23. The method of claim 22, wherein said cancer is selected from the group consisting of lung, breast, ovary, prostate, colon, liver, gastrointestinal, head-and-neck, cervix, kidney, neuroblastoma, leukemia, lymphoma, and melanoma.

24. The method of claim 22, wherein said composition is configured to be administered to a patient with a pharmaceutically acceptable carrier selected from the group consisting of an additive, a diluent, a solvent, a filter, a lubricant, an excipient, a binder, a stabilizer and combinations thereof.

25. The method of claim 22, wherein said composition is administered using a method selected from the group consisting of inhalation spray, orally, parenterally, topically, rectally, nasally, buccally, vaginally and via an implanted reservoir.

26. The method of claim 22, wherein said composition is administered using a parenteral administration selected from the group consisting of subcutaneous, intracutaneous, intravenous, intramuscular, intraperitoneal, intraarticular, intrasynovial, intrasternal, intrathecal, intralesional and intracranial injection and infusion techniques.
